(19) United States
(12) Patent Application Publication
(10) Pub. No.: US 2026/0077136 A1
(43) Pub. Date: Mar. 19, 2026

(54) Medicament delivery system

(71) Applicant: SHL Medical AG, Zug (CH)

(72) Inventors: Nurettin Ali, Deerfield Beach, FL (US); Iskender Okan Ates, Deerfield Beach, FL (US)

(21) Appl. No.: 19/110,482

(22) PCT Filed: Sep. 1, 2023
(86) PCT No.: PCT/EP2023/074088

§ 371 Date: Mar. 11, 2025

Related U.S. Application Data
(60) Provisional application No. 63/406,100, filed on Sep. 13, 2022.

(30) Foreign Application Priority Data
Nov. 14, 2022 (EP) 22207098.9

(51) Int. Cl. A61M 5/32; A61M 5/20

(52) U.S. Cl. CPC A61M 5/3204; A61M 5/20; A61M 2205/586

(57) ABSTRACT

The present disclosure relates to a medicament delivery system comprising a medicament delivery device comprising a needle and a needle shield, wherein a proximal end of the medicament delivery device is directed towards an injection site and a distal end of the medicament delivery device is directed away from the injection site, and a cap assembly, wherein the cap assembly comprises: a central axis, and at least two grip means. The at least two grip means extend in the direction of the central axis and are arranged around the central axis to form circular segments. A distal end of each of the at least two grip means is bent towards the central axis to form a hook, wherein each hook is configured to wedge a distal edge of the needle shield. The disclosure further relates to a corresponding cap assembly and a corresponding method.

The present disclosure relates to a medicament delivery system, a cap assembly for a medicament delivery system and a method for preparing a medicament delivery system for use.

Medicament delivery systems, in particular autoinjector systems, according to the prior art usually comprise caps configured to remove a needle shield before use of the device. Depending on the type of needle shield, e.g., a rather flexible rubber needle shield (FNS) or a rigid needle shield (RNS), different mechanisms are provided to de-cap the device. However, a safe handling and disposal of the cap and the needle shield may not always be ensured.

The present disclosure has thus been made in view of the above problems and mentioned deficiencies.

The invention is defined in the independent claims. Dependent claims describe preferred embodiments.

The present disclosure relates to a medicament delivery system comprising a medicament delivery device comprising a needle and a needle shield, wherein a proximal end of the medicament delivery device is directed towards an injection site and a distal end of the medicament delivery device is directed away from the injection site, and a cap assembly, wherein the cap assembly comprises: a central axis, and at least two grip means. The at least two grip means extend in the direction of the central axis and are arranged around the central axis to form circular segments. A distal end of each of the at least two grip means is bent towards the central axis to form a hook, wherein each hook is configured to wedge a distal edge of the needle shield.

Various embodiments may preferably implement the following features:

Preferably, each distal end of the at least two grip means is tilted towards the central axis. This structure allows a better gripping and wedging of the needle shield while avoiding any unintended interference with other elements of the medicament delivery device.

Preferably, the needle shield comprises an outer needle shield and an inner needle shield with respect to the central axis.

Preferably, the hooks are configured to engage the needle shield between the inner and the outer needle shield to wedge the outer needle shield.

Preferably, the outer needle shield is a rigid needle shield, and the inner needle shield is a flexible needle shield.

Preferably, during attachment of the cap assembly to the proximal end of the medicament delivery device, the at least two grip means are configured to be bent outwardly with respect to the central axis around the needle shield and to flex inwardly after the distal edge of the needle shield is passed. The hooks are preferably configured to wedge the needle shield at the distal edge of the needle shield when the cap assembly is removed in a proximal direction such that the cap assembly and the needle shield are removed together. Preferably, The grip means are flexible or deflectable means which are configured to flex outwardly and inwardly with in a radial direction with respect to the central axis.

Preferably, the hooks are configured to hold the needle shield in the cap assembly.

Preferably, each grip means comprises at least three flexible support ribs. Preferably, the flexible support ribs of each grip means are arranged parallel to each other in the direction of the central axis and are connected to each other by the hook of the corresponding grip means. The support ribs can provide flexibility in the direction perpendicular to the central axis, i.e. in a radial direction, while ensuring a sufficient rigidity for wedging and removing the needle shield.

The present disclosure further relates to a cap assembly for removing a needle shield from a medicament delivery device, wherein the cap assembly comprises a central axis and at least two grip means. The at least two grip means extend in the direction of the central axis and are arranged around the central axis to form circular segments. An end of the grip means is bent towards the central axis to form a hook for wedging an edge of the needle shield.

The circular segments may be disposed rotationally symmetric around the central axis.

The hook may be shaped to have a curvature of equal to or less than 180° and to have dimensions suitable for engaging the edge of the needle shield.

The hooks may also be described as (quasi-) semicircle clips or wedging clips. That is, the hooks are preferably tapered in a distal direction to be able to wedge (and preferably hold) the edge of the needle shield, when the hooks are pulled or pushed onto said edge.

Preferably, the cap assembly is configured to be used in a system as described above.

The cap assembly preferably comprises a proximal, outer cylindrical portion configured to be gripped by a patient to handle or remove the cap assembly from the device.

The cap assembly may comprise an inner cylindrical portion connected to the outer cylindrical portion or integrally formed with the outer cylindrical portion. The inner and outer cylindrical portions may be disposed concentrically around a central axis of the cap assembly.

The at least two grip means are preferably provided at the distal end of the inner cylindrical portion. Alternatively, the grip means may also form the inner portion, i.e., extend from the proximal end of the cap assembly.

The present disclosure also relates to a method for preparing a medicament delivery system as described above for use. The method comprises attaching the cap assembly to the proximal end of the medicament delivery device and removing the cap assembly together with the needle shield.

Various embodiments may preferably implement the following features.

Preferably, during attaching the cap assembly the at least two grip means bend outwardly with respect to the central axis around the needle shield and flex inwardly after a distal edge of the needle shield is passed. Preferably, the grip means wedge the needle shield at the distal edge of the needle shield when the cap assembly is removed in a proximal direction such that the cap assembly and the needle shield are removed together and hold the needle shield in the cap assembly.

The disclosure is further described with reference to the following figures.

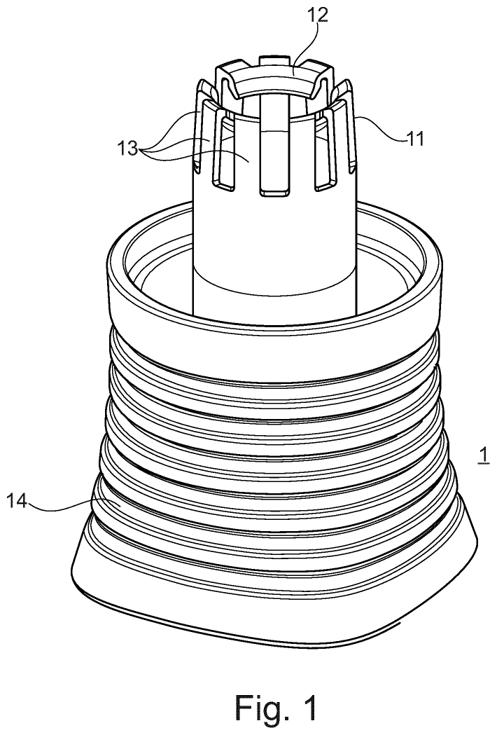

FIG. 1 shows a perspective view of a cap assembly according to an embodiment.

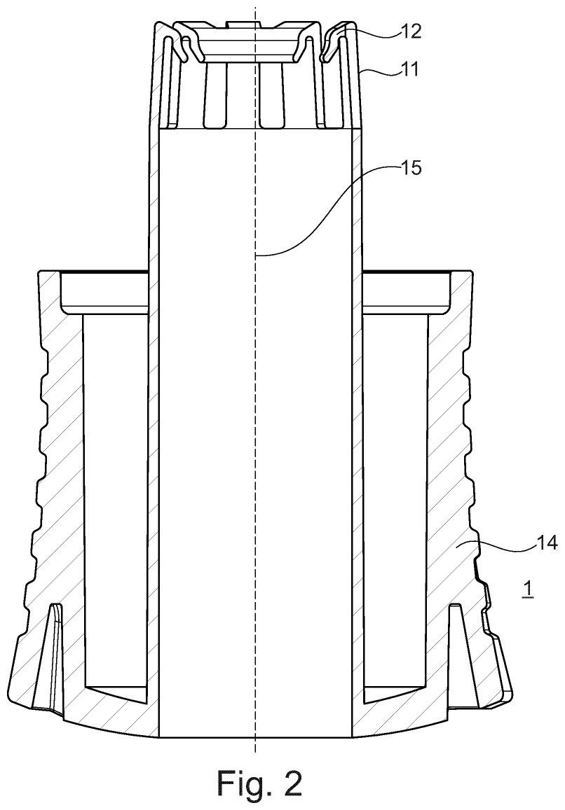

FIG. 2 shows a sectional view of a cap assembly according to an embodiment.

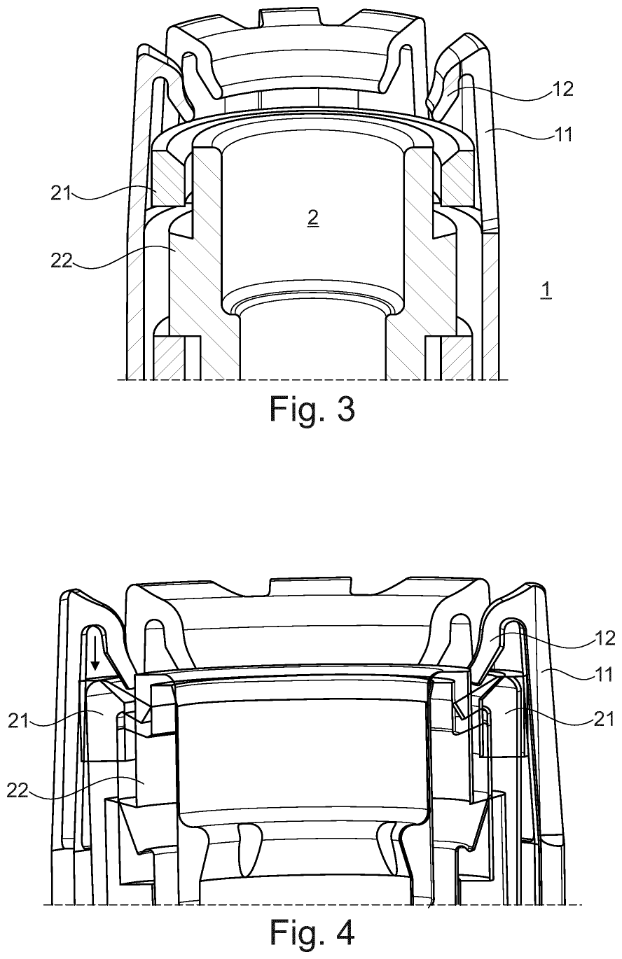

FIG. 3 shows an enlarged sectional view of a cap assembly according to an embodiment.

FIG. 4 shows an enlarged sectional view of a cap assembly and a needle shield according to an embodiment.

In the following, the terms cap and cap assembly may be used interchangeably. The cap assembly may be integrally formed or may comprise several elements which are connected to each other, e.g. by gluing, snap-fit or the like. The cap may be formed of a synthetic material.

Moreover, the term proximal refers to the direction towards an injection site and the term distal to a direction away from an injection site.

FIG. 1 shows a perspective view of a cap assembly 1 according to an embodiment. The cap assembly 1 may comprise a proximal, outer cylindrical portion 14 configured to be gripped by a patient to handle or remove the cap assembly from the device. The term cylindrical is not to be understood as limiting and only denotes a general shape of the element(s). The grip portion 14 of FIG. 1 is slightly conical. The cap assembly may also have a rectangular shape. The exemplary cap assembly according to FIG. 1 further comprises an inner cylindrical portion connected to the outer cylindrical portion or integrally formed with the outer cylindrical portion. The inner and outer cylindrical portions may be disposed concentrically around a central axis 15 (see FIG. 2) of the cap assembly 1.

At the distal end of the inner cylindrical portion, at least two grip means 11 are provided. Alternatively, the grip means 11 may also form the inner portion, i.e., extend from the proximal end of the cap assembly 1. The grip means 11 are flexible or deflectable means which are configured to flex outwardly and inwardly with in a radial direction with respect to the central axis.

The cap assembly 1 may be used in a medicament delivery system comprising a medicament delivery device. The medicament delivery device comprises a needle and a needle shield 2, wherein a proximal end of the medicament delivery device is directed towards an injection site and a distal end of the medicament delivery device is directed away from the injection site.

As outlined above, the cap assembly 1 comprises a central axis 15 and at least two grip means 11. The at least two grip means 11 extend in the direction of the central axis 15 and are arranged around the central axis 15 to form circular segments. The circular segments may be disposed rotationally symmetric around the central axis 15. A distal end of the at least two grip means 11 is bent towards the central axis 15 to form a hook 12, wherein the hook 12 is configured to wedge a distal edge of the needle shield 2 (not shown). In particular, the hook 12 may be shaped to have a curvature of equal to or less than 180° and to have dimensions suitable for engaging the edge of the needle shield 2. The hooks 12 may also be described as (quasi-) semicircle clips or wedging clips. That is, the hooks 12 are preferably tapered in a distal direction to be able to wedge (and preferably hold) the edge of the needle shield 2, when the hooks 12 are pulled or pushed onto said edge.

The structure of the hooks 12 can also be seen from FIG. 2 showing a sectional view of an exemplary cap assembly 1 according to the present disclosure. In this view. the central axis 15 is indicated by a dashed line. Particularly, each distal end of the at least two grip means 11 is tilted towards the central axis. This structure allows a better gripping and wedging of the needle shield 2 while avoiding any unintended interference with other elements of the medicament delivery device.

FIG. 3 is a further enlarged sectional view of a cap assembly 1 according to an exemplary embodiment, wherein a needle shield 2 is located inside the cap assembly. The medicament delivery device and the syringe/needle on which the needle shied 2 is placed is not shown in this figure. According to FIG. 3, the needle shield comprises an outer needle shield 21 and an inner needle shield 22. The terms outer and inner herein refer to their respective distance to the central axis 15, wherein the outer needle shield 21 is further away from the central axis 15 than the inner needle shield 22. The outer needle shield 21 and the inner needle shield 22 are connected such that they can be moved together. The hooks 12 may be configured to engage the needle shield 2 between the inner 22 and the outer needle shield 21 to wedge the outer needle shield 21. The outer needle shield 21 may be a rigid needle shield, RNS, and the inner needle shield 22 may be a flexible needle shield, FNS.

Each grip means 11 may be formed of plastic and may comprise at least two, particularly at least three flexible support ribs 13 as shown in the figures. The flexible support ribs 13 of each grip means are arranged parallel to each other in the direction of the central axis 15 and connected by the respective hooks 12. The support ribs 13 provide a flexibility in the direction perpendicular to the central axis 15, i.e. in a radial direction, while ensuring a sufficient rigidity for wedging and removing the needle shield 2.

The grip means 11 each form a circular section. The additional support rib 13 structure allow the grip means to be more flexible and shorter at the same time. If the grip means 11 were wider, such as a complete semicircle, the grip means 11 need to be longer to provide enough flexibility for inserting a needle shield 2 in between. Therefore, the length of the grip means 11, the width of the circular section forming the grip means 11 as well as the width and number of the support ribs 13, if any, can be used to control the flexibility and rigidity of the grip means 11.

As mentioned above, the grip means 11 and thus the flexible support ribs 13 may be tilted inwards. The hooks 12, which are integrally formed with the grip means 11 and thus the support ribs 13 may also themselves be flexible. Hence, the hooks 12 may slightly spread when they are pushed or pulled onto the distal edge of the needle shield 2, in particular the outer needle shield 21. The flexibility of the hooks may further aid in wedging (and holding) the needle shield 2.

Attachment of the cap assembly 1 onto a medicament delivery device having a needle shield 2 placed on the needle is performed as follows. During attachment of the cap assembly 1 to the proximal end of the medicament delivery device, the at least two grip means 11 are configured to be bent outwardly with respect to the central axis 15 around the needle shield 2. As explained above, the grip means 11 and, if applicable, the support ribs 13, respectively, are flexible in a radial direction. After the distal edge of the needle shield 2 is passed, the grip means 11 are allowed to flex back inwardly towards the central axis 15 and above the distal edge of the needle shield 2. The cap assembly 1 is then in a position as shown in FIG. 3, in which the grip means 11 and the hooks 12, respectively, have not yet engaged or wedged the edge of the needle shield 2. In other words, the flexibility of the grip means 11 allows the grip means to flex and open and allows the needle shield 2 to go through when the cap assembly 1 is attached to a medicament delivery device.

When the cap assembly 1 is subsequently removed in a proximal direction to expose the needle before use, the hooks 12 are configured to wedge the needle shield 2 at the distal edge of the needle shield 2. In other words, the hooks 12 apply a force onto both (opposing) sides of the sheath walls. Thus, the hooks 12 apply a force in the radial direction on the edge of the outer needle shield 21, i.e., an outer and inner wall of the outer needle shield 21. Moreover, the pulling movement applies a force on the needle shield 2 in the proximal direction allowing the needle shield 2 to also be moved in the proximal direction. Thereby, the cap assembly 1 and the needle shield 2 are removed together. The hooks 12 are particularly configured to hold the needle shield 2 in the cap assembly 1. In this manner, the cap assembly 1 and the needle shield 2 can be disposed of together or the cap assembly 1 and the needle shield 2 may be re-attached to the medicament delivery device. The wedged needle shield 2 may also be manually removed from the hooks 12. The cap assembly 1, i.e. the inner cylindrical portion, may particularly be open in a proximal direction to allow removal of the needle shield 2 (see e.g. FIG. 2).

FIG. 4 shows an enlarged sectional view of a cap assembly 1 having a needle shield 2 placed therein, i.e. being placed on the proximal end of a medicament delivery device (the medicament delivery device, syringe and needle are not shown). As can be seen from FIG. 4, when the cap assembly 1 is moved in the proximal direction (downwards in FIG. 4) as indicated by the arrow, the hooks 12 engage between the outer needle shield 21 and the inner needle shield 22 to wedge the outer needle shield 21. As described above, the flexibility of the grip means 11 and the hooks 12 aids in wedging the edge of the outer needle shield 21. The needle shield 2 is thus also retained in the cap assembly 1 after removal from the medicament delivery device.

The present disclosure also relates to a cap to be used with the above described medicament delivery system. In particular and as described above, the cap assembly 1 for removing a needle shield 2 from a medicament delivery device comprises a central axis 15 and at least two grip means 11, wherein the at least two grip means 11 extend in the direction of the central axis 15 and are arranged in a rotationally symmetric manner around the central axis 15. An end of the grip means 11 is bent towards the central axis 15 to form a hook 12 for wedging an edge of the needle shield 2.

The present disclosure further relates to a method for assembling a medicament delivery system as described above. The method may comprise removing the cap assembly 1 from the medicament delivery system, i.e. the present disclosure may relate to a method for using the medicament delivery system. In particular, the present disclosure may refer to a method for preparing a medicament delivery system or medicament delivery device for use.

The method comprises attaching the cap assembly 1 to the proximal end of the medicament delivery device and removing the cap assembly 1 together with the needle shield 2.

During attachment of the cap assembly 1, the at least two grip means 11 bend outwardly with respect to the central axis 15 around the needle shield 2 and flex inwardly after a distal edge of the needle shield 2 is passed. The grip means 11 wedge the needle shield 2 at the distal edge of the needle shield 2 when the cap assembly 1 is removed in a proximal direction such that the cap assembly 1 and the needle shield 2 are removed together. Particularly, the grip means 11 hold the needle shield 2 in the cap assembly 1.

Hence, the present disclosure can provide a safe and reliable needle shield removal mechanism which can allow efficient medicament delivery device assembly and can allow the needle shield to be wedged inside the cap for removal. Moreover, the needle shield may be retained in the cap for disposal or re-attachment.

The delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Other aspects, features, and advantages will be apparent from the summary above, as well as from the description that follows, including the figures and the claims.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below.

Furthermore, in the claims the word “comprising” does not exclude other elements or steps, and the indefinite article “a” or “an” does not exclude a plurality. A single unit may fulfil the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

1-11. (canceled)

12. A medicament delivery system comprising:
a medicament delivery device comprising a needle and a needle shield, wherein a proximal end of the medicament delivery device is directed towards an injection site and a distal end of the medicament delivery device is directed away from the injection site; and
a cap assembly, wherein the cap assembly comprises:
a central axis; and
at least two grip means,
wherein the at least two grip means extend in the direction of the central axis and are arranged around the central axis to form circular segments, and
wherein a distal end of each of the at least two grip means is bent towards the central axis to form a hook, wherein each hook is configured to wedge a distal edge of the needle shield.

13. The medicament delivery system according to claim 12, wherein each distal end of the at least two grip means is tilted towards the central axis.

14. The medicament delivery system according to claim 12,
wherein the needle shield comprises an outer needle shield and an inner needle shield with respect to the central axis and
wherein the hooks are configured to engage the needle shield between the inner and the outer needle shield to wedge the outer needle shield.

15. The medicament delivery system according to claim 14, wherein the outer needle shield is a rigid needle shield, and the inner needle shield is a flexible needle shield.

16. The medicament delivery system according to claim 11,
wherein, during attachment of the cap assembly to the proximal end of the medicament delivery device, the at least two grip means are configured to be bent outwardly with respect to the central axis around the needle shield and to flex inwardly after the distal edge of the needle shield is passed, and
wherein the hooks are configured to wedge the needle shield at the distal edge of the needle shield when the cap assembly is removed in a proximal direction such that the cap assembly and the needle shield are removed together.

17. The medicament delivery system according to claim 11, wherein the hooks are configured to hold the needle shield in the cap assembly.

18. The medicament delivery system according to claim 11,
wherein each grip means comprises at least three flexible support ribs, and
wherein the flexible support ribs of each grip means are arranged parallel to each other in the direction of the central axis and are connected to each other by the hook of the corresponding grip means.

19. A cap assembly for removing a needle shield from a medicament delivery device, wherein the cap assembly comprises:
a central axis; and
at least two grip means,
wherein the at least two grip means extend in the direction of the central axis and are arranged around the central axis to form circular segments,
wherein an end of the grip means is bent towards the central axis to form a hook for wedging an edge of the needle shield.

20. The cap assembly according to claim 8, wherein the cap assembly is configured to be used in a system according to any one of claims 1 to 7.

21. A method for preparing the medicament delivery system according to claim 11 for use, the method comprising:
attaching the cap assembly to the proximal end of the medicament delivery device, and
removing the cap assembly together with the needle shield.

22. The method according to claim 21, wherein during attaching the cap assembly the at least two grip means bend outwardly with respect to the central axis around the needle shield and flex inwardly after a distal edge of the needle shield is passed, and
wherein the grip means wedge the needle shield at the distal edge of the needle shield when the cap assembly is removed in a proximal direction such that the cap assembly and the needle shield are removed together and hold the needle shield in the cap assembly.
